(19) United States
(12) Patent Application Publication
(10) Pub. No.: US 2009/0285893 A1
(43) Pub. Date: Nov. 19, 2009

(54) TREATMENT OF HEART FAILURE IN WOMEN

(75) Inventors: Roger Karam, Cincinnati, OH (US); Giuseppe Massimo Claudio Rosano, Roma (IT); Maurizio Volterrani, Monticelli Brusati (IT)

Correspondence Address: COVINGTON & BURLING, LLP;ATTN: PATENT DOCKETING, 1201 PENNSYLVANIA AVENUE, N.W., WASHINGTON, DC, 20004-2401, US

(73) Assignee: The Procter & Gamble Company, Cincinnati, OH (US)

(21) Appl. No.: 12/465,467

(22) Filed: May 13, 2009

Related U.S. Application Data
(60) Provisional application No. 61/071,810, filed on May 19, 2008.
(60) Provisional application No. 61/071,811, filed on May 19, 2008.

(51) Int. Cl. A61K 9/10; A61K 31/56; A61P 9/04

(52) U.S. Cl. 424/484; 514/178

(57) ABSTRACT

A method is disclosed utilizing an androgen such as testosterone and/or a selective androgen receptor modulator for treating or delaying the further development of heart failure, and other disorders in females including manifestations of heart failure and concomitant cardiovascular and noncardiovascular disorders.

### CROSS-REFERENCE TO RELATED APPLICATIONS

This application claims the benefit under 35 U.S.C. § 119(e) of U.S. Provisional Patent Application No. 61/071,810, filed May 19, 2008, herein incorporated by reference in its entirety, and claims the benefit under 35 U.S.C. § 119(e) of U.S. Provisional Patent Application No. 61/071,811, filed May 19, 2008, herein incorporated by reference in its entirety.

### BACKGROUND OF THE INVENTION

Heart failure is a very significant public health concern. In particular, it is a major and growing problem in the United States. Approximately five million people in the United States have heart failure. Hunt S A, Abraham W T, Chin M H, Feldman A M, Francis G S, Ganiats T G, Jessup M, Konstam M A, Mancini D M, Michl K, Oates J A, Rahko P S, Silver M A, Stevenson L W, Yancy C W. ACC/AHA 2005 guideline update for the diagnosis and management of chronic heart failure in the adult: summary article: a report of the American College of Cardiology/American Heart Association Task Force on Practice Guidelines (Writing Committee to Update the 2001 Guidelines for the Evaluation and Management of Heart Failure). J Am Coll Cardiol 2005; 46:1116-43 (“ACA/AHA Practice Guideline”). Each year, more than 550,000 individuals are diagnosed with heart failure for the first time. Id.

An analysis of data from the National Hospital Discharge Survey from 1990 to 1999 indicates that hospitalizations for heart failure have continued to rise. Koelling, et al., “The Expanding National Burden of Heart Failure in the United States: The influence of Heart Failure in Women,” Am. Heart J. 147(1):76 (2003) (“Koelling”). One explanation for the continued increase in heart failure hospitalizations is the increasing number of heart failure hospitalizations of females. Id. The increased age-adjusted heart failure hospitalization rate for females accounted for nearly half of the total increase in heart failure hospitalizations over the ten-year period. Id. In fact, one cause of heart failure, coronary artery disease, is the single largest killer of American females. American Heart Association, “Heart Disease and Stroke Statistics-2005 Update.” Dallas, Tex.: American Heart Association (2005).

Current drugs used in individuals with heart failure include diuretics, digoxin, angiotensin-converting enzyme inhibitors (“ACEIs”), angiotensin II receptor blockers (“ARBs”), beta-blockers and digitalis. Many current heart failure drugs cause adverse side effects. For example, there are risks involved with drugs such as ARBs and ACEIs. Such risks include angioedema, hypotension, renal dysfunction and hyperkalemia. These risks are greater when such drugs are combined together, i.e., with another inhibitor of the same metabolic pathway. Also, some drugs used to treat heart failure can cause or exacerbate pulmonary symptoms. For example, ACEIs can cause cough, and beta-blockers can aggravate bronchospastic symptoms in patients with asthma.

In addition, it has been found that some current drugs used for heart failure such as digoxin and digitalis are disadvantageous in females. For example, it has been reported that the efficacy of digoxin in females for heart failure is unclear. ACA/AHA Practice Guideline at 1136. In addition, an increase in the risk of mortality in females treated with digitalis has been reported. Koelling at 77.

Accordingly, it would be particularly desirable to find efficacious methods of treatment of heart failure in female patients without aggravating the aforementioned adverse effects of available treatment.

### BRIEF SUMMARY OF THE INVENTION

It has now been discovered that androgens are effective compounds that are useful in females for treating heart failure, manifestations of heart failure, and concomitant disorders, or slowing or ceasing the progression of heart failure, manifestations of heart failure, and concomitant disorders. Additionally, selective androgen receptor modulators, which bind select androgen receptors without exhibiting side effects associated with androgens, can also be used for treating heart failure or slowing or ceasing the progression of heart failure.

The present invention involves treating heart failure in females with an androgen, such as testosterone, and/or a selective androgen receptor modulator (“SARM”) or slowing or ceasing the progression of heart failure in females with an androgen, such as testosterone, and/or SARM. The present invention also relates to treating, or slowing or ceasing the progression of, heart failure in females having a structural heart change or structural heart changes by administering a therapeutically effective amount of an androgen, such as testosterone, and/or SARM. The invention further relates to treating, or slowing or ceasing the progression of, heart failure in females with coronary artery disease, hypertension or cardiomyopathy by administering a therapeutically effective amount of an androgen and/or SARM. The invention also relates to treating, in females with heart failure, manifestations of heart failure, including exercise intolerance and insulin resistance, by the administration of a therapeutically effective amount of an androgen and/or SARM.

The present invention moreover relates to use of an androgen, such as testosterone, and/or SARM in the manufacture of a medicament for treating heart failure in females or for slowing or ceasing the progression of heart failure in females. The present invention also relates to use of an androgen, such as testosterone, and/or SARM in the manufacture of a medicament for treating heart failure in females or for slowing or ceasing the progression of heart failure in females having a structural heart change or structural heart changes. In addition, the invention relates to use of an androgen, such as testosterone, and/or SARM in the manufacture of a medicament for treating, or for slowing or ceasing the progression of, heart failure in females with coronary artery disease, hypertension or cardiomyopathy. The present invention further relates to use of an androgen, such as testosterone, and/or SARM in the manufacture of a medicament for treating, in females with heart failure, manifestations of heart failure, including exercise intolerance and insulin resistance.

The present invention additionally relates to an androgen, such as testosterone, and/or SARM for use in treating heart failure in females or in slowing or ceasing the progression of heart failure in females. The present invention also relates to an androgen, such as testosterone, and/or SARM for use in treating heart failure in females or in slowing or ceasing the progression of heart failure in females having a structural heart change or structural heart changes. The invention further relates to an androgen, such as testosterone, and/or SARM, for use in treating, or in slowing or ceasing the progression of, heart failure in females with coronary artery disease, hypertension or cardiomyopathy. The present invention further relates to an androgen, such as testosterone, and/or SARM for use in treating, in females with heart failure, manifestations of heart failure, including exercise intolerance and insulin resistance.

### BRIEF DESCRIPTION OF THE DRAWINGS

FIG. 1 shows exercise tolerance results (in meters) for a 6 minute walking test (“6MWT”) for testosterone and placebo groups at baseline, three months and six months.

FIG. 2 shows exercise tolerance results (in Δ meters) for a 6MWT test with a comparison between groups at three months and six months for testosterone and placebo groups.

FIG. 3 shows exercise tolerance results (percent changes from baseline) for a 6MWT at three months and six months for testosterone and placebo groups.

FIG. 4 shows the same data as FIG. 1 in a comparison between groups at baseline, three months and six months for testosterone and placebo groups.

FIG. 5 shows exercise tolerance results for maximal oxygen consumption peak (Ml/kg/min) at baseline, three months and six months for testosterone and placebo groups.

FIG. 6 shows a between-groups comparison of exercise tolerance results for maximal oxygen consumption peak (percent changes from baseline) at three months and six months for testosterone and placebo groups.

FIG. 7 shows the same data as FIG. 5 in a comparison between groups at baseline, three months and six months for testosterone and placebo groups.

FIG. 8 shows muscle isometric strength results (Newton) for maximal voluntary contraction (“MVC”) at baseline, three months and six months for testosterone and placebo groups.

FIG. 9 shows muscle isometric strength results for maximal voluntary contraction (“MVC”) (percent changes from baseline) at three months and six months for testosterone and placebo groups.

FIG. 10 shows the same data as FIG. 8 in a comparison between groups at baseline, three months and six months for testosterone and placebo groups.

FIG. 11 shows muscle isokinetic strength results for peak torque (Newton/m) at baseline, three months and six months for testosterone and placebo groups.

FIG. 12 shows muscle isokinetic strength results for peak torque (percent changes from baseline) at three months and six months for testosterone and placebo groups.

FIG. 13 shows the same data as FIG. 11 in a comparison between groups at baseline, three months and six months for testosterone and placebo groups.

FIG. 14 shows improvement in New York Heart Association (“NYHA”) classification from NYHA Class III to Class II after three and six months of treatment with testosterone and with placebo.

FIG. 15 shows improvement of NYHA Class and/or improvement of >15% 6MWT at six months for testosterone and placebo groups.

FIG. 16 shows an evaluation of changes in insulin resistance between 6 months and baseline in testosterone and placebo groups using the homeostasis model assessment (“HOMA”) index (HOMA index: Fasting glycemia×fasting insulinemia/22.5).

FIG. 17 shows endothelial-dependent vasodilation results at baseline, three months and six months for one patient who received testosterone treatment.

FIG. 18 shows HDL percent changes at three months and at six months for testosterone and placebo groups.

FIG. 19 shows effect on HDL levels (in mg/dL) in a comparison between groups at baseline, three months and six months for testosterone and placebo groups.

### DETAILED DESCRIPTION OF THE INVENTION

The present invention encompasses a method of treating heart failure, or slowing or ceasing the progression of heart failure, in human females which comprises administering a therapeutically effective amount of an androgen and/or SARM.

Heart failure is a clinical syndrome that is characterized by specific symptoms and signs. Heart failure can result from any structural or functional cardiac disorder that impairs the ability of a ventricle to fill with or eject blood. A diagnosis of heart failure is in large part a clinical diagnosis that is based on a careful history and physical examination. Heart failure can be minimally symptomatic and escape diagnosis.

Heart failure can be classified in a patient by the degree of functional capacity and objective assessment within the New York Heart Association (“NYHA”) classification. Table 1 shows the NYHA Classification.¹

TABLE 1

| Functional Capacity | Objective Assessment |
|---|---|
| Class I. Patients with cardiac disease but without resulting | A. No objective |
| limitation of physical activity. Ordinary physical activity does | evidence of |
| not cause undue fatigue, palpitation, dyspnea, or anginal pain. | cardiovascular disease. |
| Class II. Patients with cardiac disease resulting in slight | B. Objective evidence |
| limitation of physical activity. They are comfortable at rest. | of minimal |
| Ordinary physical activity results in fatigue, palpitation, | cardiovascular disease. |
| dyspnea, or anginal pain. | |
| Class III. Patients with cardiac disease resulting in marked | C. Objective evidence |
| limitation of physical activity. They are comfortable at rest. | of moderately severe |
| Less than ordinary activity causes fatigue, palpitation, | cardiovascular disease. |
| dyspnea, or anginal pain. | |
| Class IV. Patients with cardiac disease resulting in inability | D. Objective evidence |
| to carry on any physical activity without discomfort. | of severe |
| Symptoms of heart failure or the anginal syndrome may be | cardiovascular disease. |
| present even at rest. If any physical activity is undertaken, | |
| discomfort is increased. | |

¹ 1994 Revisions to Classification of Functional Capacity and Objective Assessment of Patients With Diseases of the Heart (Apr. 8, 2008), at American Heart Association internet website/identifier = 4569 (citing The Criteria Committee of the New York Heart Association, Nomenclature and Criteria for Diagnosis of Diseases of the Heart and Great Vessels 253-256 (9th ed., Little, Brown & Co 1994) (1928)).

Heart failure can be a progressive disorder. The development of heart failure can be classified in four stages, which are identified as Stages A-D. ACA/AHA Practice Guideline at 1118; 1120; 1121. The ACA/AHA classification recognizes that heart failure can have established risk factors and structural prerequisites. Stage A refers to patients at high risk for heart failure but without structural heart disease or symptoms of heart failure, and Stage B involves patients with structural heart disease but without signs or symptoms of heart failure. Id. Thus, patients in Stages A and B are those individuals with risk factors that clearly predispose toward the development of heart failure. Id. Stage C refers to patients with structural heart disease with current or past symptoms of heart failure, and Stage D involves refractory heart failure that might require specialized treatment. Id.

The present invention is directed to treating heart failure, or slowing or ceasing the progression of heart failure, in subjects exhibiting structural heart disease or changes. The subject may have current or past symptoms of heart failure, but the symptoms alone are not enough as the subject must have one or more structural heart changes. The structural heart changes may be an enlarged heart or other structural changes to the heart.

As used herein, “treating heart failure” may include improving a subject's heart health. As used herein, the phrase “delaying the further development of heart failure” involves slowing or ceasing the progression of heart failure in subjects with heart failure. The term “or,” as used herein, denotes alternatives that may, where appropriate, be combined; that is, the term “or” includes each listed alternative separately as well as their combination. As used herein, unless the context clearly dictates otherwise, references to the singular, such as the singular forms “a,” “an,” and “the,” include the plural, and references to the plural include the singular.

One embodiment of the present invention involves treating or delaying the further development of heart failure with a pharmaceutically effective amount of an androgen and/or SARM in females classified in Stage B, C or D ACA/AHA classification of heart failure. A further embodiment of the invention involves improving a female's ACA/AHA classification by the administration of a pharmaceutically effective amount of an androgen and/or SARM. An additional embodiment of the invention involves treating or delaying the further development of heart failure with a pharmaceutically effective amount of an androgen and/or SARM in females classified in Class III or IV NYHA Classification of heart failure.

A further exemplary aspect of the present invention involves treating or delaying the further development of heart failure in a female with a pharmaceutically effective amount of an androgen and/or SARM. A further embodiment of the invention includes delaying the further development of heart failure in a female in one of Stages B, C or D comprising the administration of an androgen and/or SARM.

The treatment of the present invention is treating heart failure syndrome and/or any of the things contributing to the syndrome in female subjects with heart failure syndrome as described herein. Further, the present invention is directed to delaying the further development of heart failure syndrome and/or any of the things contributing to the syndrome in females with heart failure syndrome as described herein.

Heart failure can result from impairment of the function of the left, right or both ventricles. Heart failure may be associated with a wide spectrum of ventricular functional abnormalities, which may range from patients with normal ventricular size and preserved cardiac pump function to those with severe heart hypertrophy or dilatation and/or markedly reduced cardiac function. Patients with heart failure may have normal or abnormal vascular endothelial function. Heart failure can be systolic or diastolic. In most patients with heart failure, abnormalities of systolic and diastolic dysfunction coexist, regardless of the vascular endothelial function. Heart failure may further result from disorders of the pericardium, myocardium, endocardium or great vessels.

Causes of heart failure include, but are not limited to, coronary artery disease, hypertension (systemic or pulmonary) and valvular heart disease (aortic, pulmonary, tricuspide and mitral valve disease). Causes of heart failure further include, but are not limited to: artherosclerosis; metabolic disorders including glycogen storage diseases, diabetes mellitus and obesity; infections, including viral infections such as HIV, bacterial infections and parasitic infections; pericardial diseases; drug toxicities, for example, doxorubicin (Adriamycin®), cyclophosphamide (Cytoxan®); drug abuse, for example, abuse of cocaine or alcohol; connective tissue disease; infiltrative diseases, for example, amyloidosis, sarcoidosis, hemochromatosis, malignancy; cardiac conduction system disorders or arrhythmias such as bradycardias and tachycardias; cardiomyopathy including obstructive cardiomyopathy; neuromuscular diseases, for example, muscular or myotonic dystrophy, Friedreich's ataxia; nutritional disorders such as beriberi and kwashiorkor; pheochromocytoma; radiation; endomyocardial fibrosis; congenital heart defects; peripartum cardiomyopathy, intracardiac shunts; atrioventricular fistula; anemia; pregnancy; Paget's disease; hyperthyroidism; dilated idiopathic cardiomyopathy; and other conditions known to cause heart failure.

Manifestations of heart failure include dyspnea and fatigue, which may limit exercise tolerance, and cause fluid retention, which may lead to pulmonary congestion and peripheral edema. Both abnormalities can impair the functional capacity and quality of life of affected individuals. Manifestations of heart failure, depending on the severity and underlying disease, include chest pain, angina, palpitations, dizziness, syncope and cardiac arrest, insulin resistance, muscle atrophy, caquexia, altered autonomic sympathetic and parasympathetic function and decreased endothelial function. Some patients have exercise intolerance but little evidence of fluid retention, whereas others refer edema and report few symptoms of dyspnea or fatigue. As not all patients have volume overload (leading to congestive symptoms) at the time of initial or subsequent evaluation, the term “heart failure” is used, rather than the term “congestive heart failure.”

The glucose insulin axis may be deranged in heart failure. (Malkin et al., “The effect of testosterone on insulin sensitivity in men with heart failure” European J. Heart Failure 9:44-50, 44 (2007).) A cross-sectional study of patients with heart failure found that about 43% of patients had manifest disorders of glucose metabolism ranging from frank diabetes to impaired glucose sensitivity. (Malkin et al. at 44.) Insulin resistance is common in patients with heart failure. Insulin resistance is a relative inability of insulin to promote glucose transport into the cells. Impaired action of insulin is inversely related to the severity of the heart failure. Accordingly, a further aspect of the invention to treat manifestations of heart failure includes treating insulin resistance.

A further exemplary embodiment of the instant invention involves treating or delaying the further development of heart failure in females having heart failure conditions or causes. A further embodiment of the invention includes treating or delaying the further development of heart failure in females having diseases such as coronary artery disease, hypertension or valvular heart disease. A still further embodiment of the invention involves treating or delaying the further development of heart failure in females having diabetes.

A further exemplary embodiment of the invention is preventing heart failure decompensation and slowing progression of disease in subjects with heart failure by the administration of a therapeutically effective amount of an androgen and/or SARM. A still further embodiment of the invention is improving, by decreasing, in subjects with heart failure signs and symptoms of heart failure, such as fatigue and shortness of breath, angina, dizziness, palpitation, edema, exercise intolerance. An embodiment of the invention also includes improving morbidity, mortality, quality of life and eventually decreasing the need for emergency room admissions and hospitalizations caused by heart failure by administering a therapeutically effective amount of an androgen and/or SARM.

An androgen is any steroid hormone that can bind an androgen receptor. Androgens include, but are not limited to, 19-carbon steroid hormones. As used herein, androgen includes any natural, synthetic or derivative androgen compound, prodrugs of androgen, precursors of androgen and metabolites of androgen. Androgens used in accordance with the present invention include, but are not limited to, testosterone-like compounds. In one aspect of the instant invention, testosterone-like compounds can be natural, for example, testosterone, dihydrotestosterone (“DHT”), or androstenedione. In a further aspect of the invention, testosterone-like compounds can be: synthetic, which includes, for example, methyl testosterone; testosterone derivatives; testosterone precursors; and testosterone prodrugs, which include testosterone propionate and testosterone undecanoate.

A SARM is a nonsteroidal androgen receptor ligand. SARMs exhibit different selectivity for target androgen receptors in different tissues. By not binding to androgen receptors in one or more tissues where undesirable side effects are produced, SARMs can avoid one or more adverse effects associated with androgens. As used herein, SARMs include any natural, synthetic or derivative nonsteroidal androgen receptor ligand, prodrugs of SARMs, precursors of SARMs and metabolites of SARMs.

As used herein, the phrases “therapeutically effective amount” and “pharmaceutically effective amount” refer to that amount of androgen and/or SARM that provides a therapeutic benefit in the treatment or management of heart failure, and in the delay in further development or progression of heart failure, and provides a therapeutic benefit in the treatment or management of manifestations associated with heart failure, such as decreased exercise tolerance, impaired endothelial function, and insulin resistance, and of concomitant cardiovascular and noncardiovascular disorders including hypertension, hyperlipidemia, diabetes mellitus and pulmonary disease.

The magnitude of a therapeutic dose of androgen and/or SARM in the management of heart failure may vary with the severity of the syndrome and the route of administration. The dose, and perhaps the dose frequency, may also vary according to the age, body weight and response of the individual patient. A therapeutic dose may include a physiological dose, sub-physiological dose or supra-physiological dose. One aspect of the invention involves a therapeutic dose that provides a serum level concentration of androgen and/or SARM similar to the physiological androgen levels detected in pre-menopause women.

Serum level concentration in an individual's blood refers to dose divided by clearance. As used herein, testosterone serum level concentration refers to the total testosterone serum level concentration, which includes naturally-occurring and delivered testosterone. In general, the total daily dose range for the conditions described herein, is a dose of androgen and/or SARM that provides a testosterone serum level concentration or a therapeutic equivalence to a testosterone serum level concentration of from about 2 ng/dL to about 2000 ng/dL or an effect equivalent to this serum range of testosterone, administered in single or divided doses administered, for example, orally, transdermally, topically, or by inhalation. Another embodiment of the invention involves a dose of androgen and/or SARM that provides a testosterone serum level concentration or a therapeutic equivalence to a testosterone serum level concentration of from about 10 ng/dL to about 500 ng/dL or an effect equivalent to this serum range of testosterone. A further aspect of the invention involves a dose of androgen and/or SARM that provides a testosterone serum level concentration or a therapeutic equivalence to a testosterone serum level concentration of from about 15 ng/dL to about 200 ng/dL or an effect equivalent to this serum range of testosterone. A still further aspect of the invention involves a transdermal dose of androgen and/or SARM that provides a testosterone serum level concentration or a therapeutic equivalence to a testosterone serum level concentration of from about 15 ng/dL to about 200 ng/dL or an effect equivalent to this serum range of testosterone. It will be apparent to one of skill in the art how to adjust the dose of androgens other than testosterone and/or of SARMs so that such androgens and/or SARMs exhibit similar biological and pharmacological activity as the doses of testosterone, and, in the case of SARMs, avoid side effects associated with testosterone.

An exemplary embodiment of the invention involves a transdermal daily dose of testosterone in the amount of about 50 mcg to about 2000 mcg. A further embodiment of the invention involves a transdermal daily dose of testosterone in the amount of about 50 mcg to about 500 mcg. A still further aspect of the invention includes a transdermal daily dose of testosterone in the amount of about 100 mcg to about 600 mcg, and a still further aspect involves a transdermal daily dose of testosterone in the amount of about 150 mcg to about 400 mcg. A further aspect of the invention involves a transdermal daily dose of testosterone of approximately 300 mcg. Another embodiment involves a transdermal daily dose of testosterone of about 50 mcg to about 500 mcg that provides about 15 ng/dL to about 200 ng/dL testosterone serum level concentration in a patient. An additional aspect involves a transdermal daily dose of testosterone of about 300 mcg that provides about 80 ng/dL to about 100 ng/dL daily testosterone serum level concentration in a patient.

Any suitable route of administration may be employed for providing the patient with an effective dosage of androgen and/or SARM according to the methods of the instant invention. For example, oral, transdermal, nasal, vaginal, rectal, parenteral, subcutaneous, intramuscular and like forms of administration may be employed. Dosage forms include patches, tablets, sprays, micronizations, injections, IVs, troches, dispersions, suspensions, solutions, capsules, gels, lotions and further like dosage forms. An exemplary embodiment of the invention is directed to a transdermal patch comprising a polymer matrix. Another exemplary embodiment involves a transdermal patch that is approximately 28 cm² comprising a polymer matrix and delivers about 300 mcg testosterone/day to a patient. Another exemplary aspect of the invention involves a transdermal patch comprising a polymer matrix and about 8.4 mg testosterone. A still further embodiment of the present invention involves a transdermal patch that is approximately 28 cm² in size comprising a polymer matrix and about 8.4 mg testosterone wherein the patch is replaced about every 3 days to about every 4 days. Another exemplary embodiment includes a transdermal patch that is approximately 28 cm² in size comprising a polymer matrix and about 8.4 mg testosterone wherein the patch is replaced approximately two times per week.

Another exemplary embodiment includes delivery as set forth in U.S. Pat. Nos. 5,460,820 and 5,780,050. A further exemplary embodiment involves the Intrinsa 300 mcg/24 hours transdermal patch, which is available in Europe.

A still further embodiment encompasses delivery of testosterone by Intrinsa, and involves a route of administration that comprises a three-layer laminate design comprising: a thin, flexible, low-density polyethylene (“LDPE”) backing film; a cast film matrix of high molecular weight acrylic adhesive containing testosterone and sorbitan monooleate; and two overlapped polyester (PET) silicon-coated release liner strips designed to be peeled off and discarded by the patient prior to applying the matrix system to their skin, wherein the patch is a 7.3 cm×4.9 cm ellipse (area=28 cm²) and delivers 300 mcg/day of testosterone. The acrylic adhesive is Duro-Tak™ 87-2888, which is a copolymer (75/25 w/w) of 2-ethylhexyl acrylate and N-vinylpyrrolidone, including a dimethylacylate crosslinker, and the adhesive comprises 85% w/w of the matrix. The active pharmaceutical ingredient is testosterone and comprises 5% w/w of the matrix. There are 8.4 mg of testosterone per 28 cm² patch. The permeation enhancer is sorbitan monooleate, a naturally occurring mixture, which is a partial oleate ester of sorbitol and its mono- and di-anhydrides. The permeation enhancer comprises 10% w/w of the matrix.

It should be understood that the phrases “therapeutically effective amount of an androgen and/or SARM” and “pharmaceutically effective amount of an androgen and/or SARM” are encompassed by the above-described dosage amounts and dose frequency schedule. It should be further understood that doses for androgens other than testosterone and for SARMs other than those specifically described herein that provide therapeutically equivalent serum level concentrations to the above-described dosages fall within the scope of the instant invention.

A further embodiment of the invention involves pharmaceutical compositions comprising an androgen, such as testosterone, and/or SARM as an active ingredient, and that also may contain a pharmaceutically acceptable carrier, and optionally, other therapeutic ingredients. A pharmaceutically acceptable carrier involves a non-toxic carrier or adjuvant that may be administered to a patient, together with one or more compounds of the present invention, and which does not destroy the pharmacological activity thereof.

The invention is further defined by reference to the following examples that further describe the method of the present invention, as well as its utility. It will be apparent to those skilled in the art that modifications, both to materials and methods, may be practiced which are within the scope of the invention.

### EXAMPLES

A double-blinded, placebo-controlled, randomized clinical trial was conducted to evaluate the effects of six (6) months of treatment with 300 mcg/day testosterone delivered by a transdermal patch in post-menopausal women with heart failure. Inclusion and major exclusion criteria are presented in Table 2. Thirty-two (32) patients enrolled in the trial. The trial was randomized to an allocation ratio of approximately 2:1 (testosterone:placebo). Twenty (20) patients received testosterone, and twelve (12) patients received placebo. Testosterone was administered to the patients as Intrinsa, which is available in Europe. Previous studies of patients who were administered Intrinsa have not shown significant increases in skeletal muscle mass of patients who were administered Intrinsa.

TABLE 2

| | |
|---|---|
| Inclusion Criteria | Female gender |
| | Post-Menopausal |
| | Left ventricular EF <40% |
| | Age >60 years |
| | Stable NYHA class III |
| Major Exclusion | Recent myocardial infarction |
| Criteria | Severe liver or kidney disease |
| | Inability to Exercise |
| | Neoplasms |

The clinical trial design involved: (A) evaluation of inclusion/exclusion criteria and study agreement; (B) visit (blood pressure, heart rate and NYHA classification); (C) six minute walking test, endothelial function, ergospirometry (when possible), muscle strength (isometric and isokinetic) and blood samples; and then (D) randomization to testosterone or placebo. The following follow-ups were conducted: six weeks (B); twelve weeks (B+C); 18 weeks (B); and 24 weeks (B+C).

Table 3 shows the population baseline characteristics of the study, including concomitant treatments. All patients had coronary artery disease and stable NYHA Class III heart failure.

TABLE 3

| | Testosterone | | Placebo | | |
|---|---|---|---|---|---|
| | N = 20 (%) | | N = 12 (%) | | P Value |
| Age, ±(SD) | 68.2 ± 6.85 | | 69.1 ± 8.6 | | Not Significant |
| | | | | | (“NS”) |
| LVEF ± (SD) | 32.3 ± 8.14 | | 33.7 ± 6.92 | | NS |
| Diabetes | 11 | (55%) | 6 | (50%) | NS |
| Hypertension | 11 | (55%) | 5 | (42%) | NS |
| Dyslipidemia | 13 | (65%) | 8 | (67%) | NS |
| Atrial fibrillation | 6 | (30%) | 3 | (25%) | NS |
| HDL ± (SD) | 36.9 ± 10.71 | | 36.9 ± 7.7 | | NS |
| HOMA ± (SD) | 2.0 ± 0.37 | | 1.9 ± 0.46 | | NS |
| Beta-blockers | 18 | (90%) | 9 | (75%) | NS |
| ACE inhibitors/ARBS | 18 | (90%) | 11 | (92%) | NS |
| Diuretics | 16 | (80%) | 9 | (75%) | NS |
| Anti-aldosterone | 12 | (60%) | 7 | (58%) | NS |
| Digoxine | 6 | (30%) | 4 | (33%) | NS |
| Antiplatelet | 20 | (100%) | 12 | (100%) | NS |
| Statins | 17 | (85%) | 8 | (70%) | NS |

For the results indicated below, statistical analyses were performed using analysis of covariance with baseline values in the model. Statistical p-values of less than 0.05 were considered significant. There was no adjustment in p-values for multiple comparisons.

### Example 1

### Exercise Tolerance

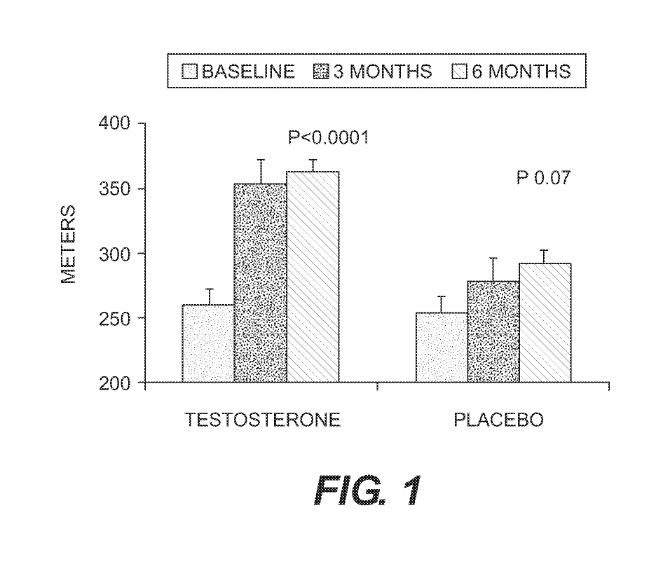

Both the testosterone group and the placebo group underwent a six minute walking test (“6MWT”) at the beginning of the study (baseline), at three months and at six months. In the 6MWT, each patient started by standing at a known beginning location point. Each patient then walked as much as possible within a 6-minute timeframe. At the end of the 6-minute timeframe, the ending location of each patient was identified. The distance each patient walked was calculated by measuring (in meters) the distance between the beginning and end points for each patient. Results are reported in Tables 4-6 and FIGS. 1-4.

TABLE 4

| | Testosterone | Placebo |
|---|---|---|
| baseline | 260 m | 254 m |
| 3 months | 353 m | 278 m |
| 6 months | 362 m | 292 m |

TABLE 5

| | 3 months | 6 months |
|---|---|---|
| Testosterone | 93 Δ m | 102 Δ m |
| Placebo | 24 Δ m | 38 Δ m |

TABLE 6

| | 3 months | 6 months |
|---|---|---|
| Testosterone | 35.7% change | 39.2% change |
| Placebo | 9.4% change | 14.9% change |

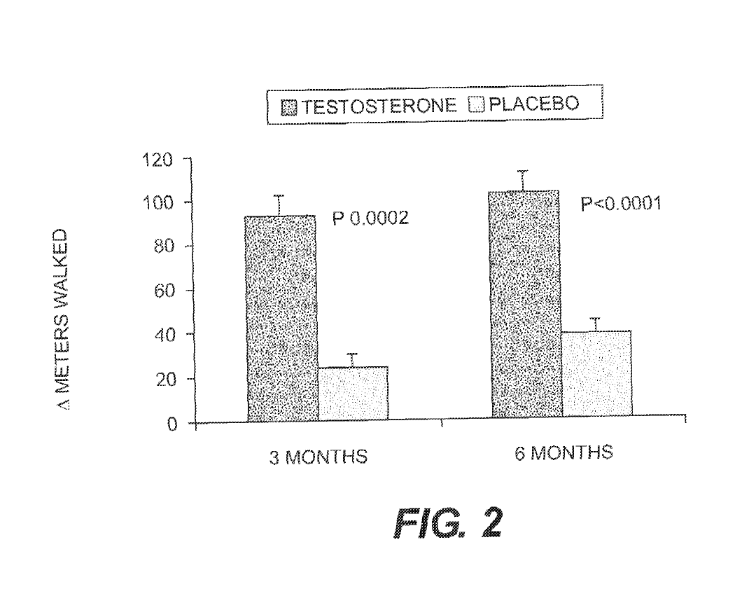

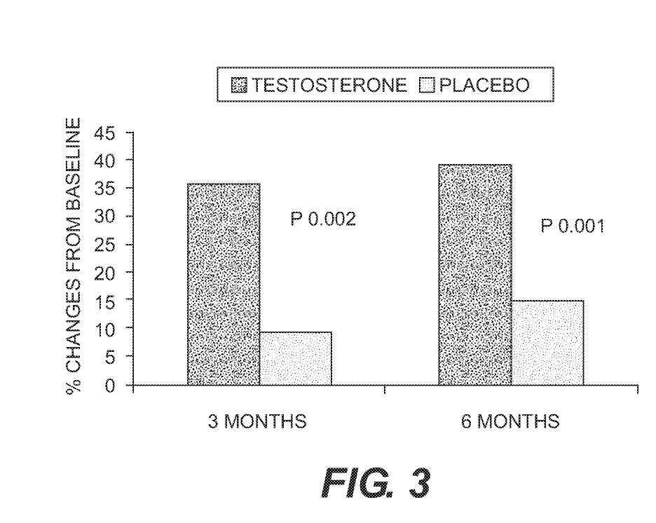

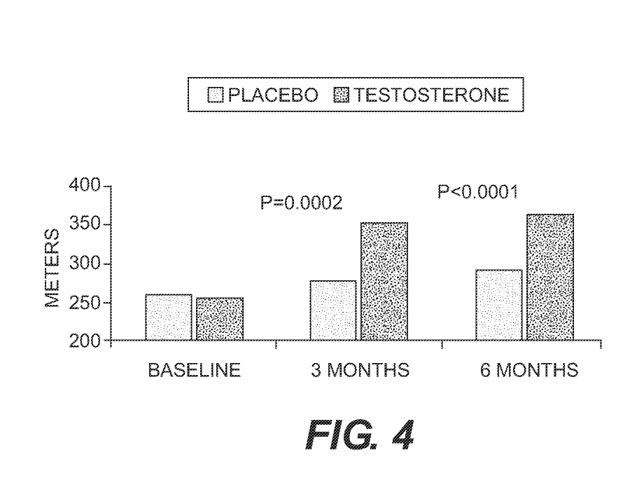

The results demonstrate a statistically significant increase in the distance walked by patients treated with testosterone, with a p-value of <0.0001 for the improvement within the testosterone group compared to baseline after six months of treatment with testosterone (FIG. 1), and p-values of 0.002 and <0.0001 for the changes shown by the testosterone group as compared to the changes in the placebo group at three and six months, respectively (FIG. 4). A statistically significant increase in the change in distance walked by patients in the testosterone group, as compared to the placebo group, occurred after treatment with testosterone at months three and six, with p-values of 0.002 and 0.001, respectively (FIG. 2). A statistically significant increase in the percent of change in distance walked by patients in the testosterone group, as compared to the placebo group, occurred after treatment with testosterone at months three and six, with p-values of 0.002 and 0.001, respectively (FIG. 3).

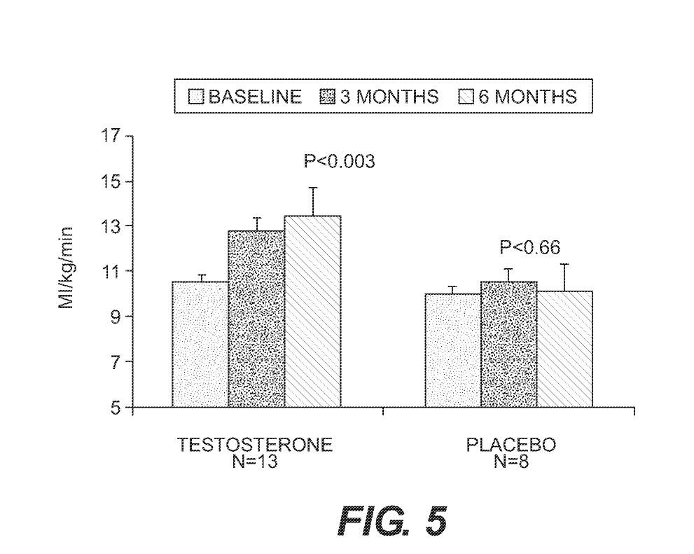

Maximal oxygen consumption (“MVO2;” also “VO2 max”) is the maximum volume of oxygen that the body can consume during intense, whole-body exercise, while breathing air at sea level. This volume is expressed as a rate, which is in ml/kg/min. As oxygen consumption is linearly related to energy expenditure, the measurement of oxygen consumption is an indirect measurement of an individual's maximal capacity to do work aerobically. Results testing MVO2 are reported at Tables 7-8 and FIGS. 5-7.

TABLE 7

| | Testosterone | Placebo |
|---|---|---|
| baseline | 10.54 ml/kg/min | 10 ml/kg/min |
| 3 months | 12.76 ml/kg/min | 10.5 ml/kg/min |
| 6 months | 13.5 ml/kg/min | 10.1 ml/kg/min |

TABLE 8

| | 3 months | 6 months |
|---|---|---|
| Testosterone | 21% change | 28% change |
| Placebo | 5% change | 1% change |

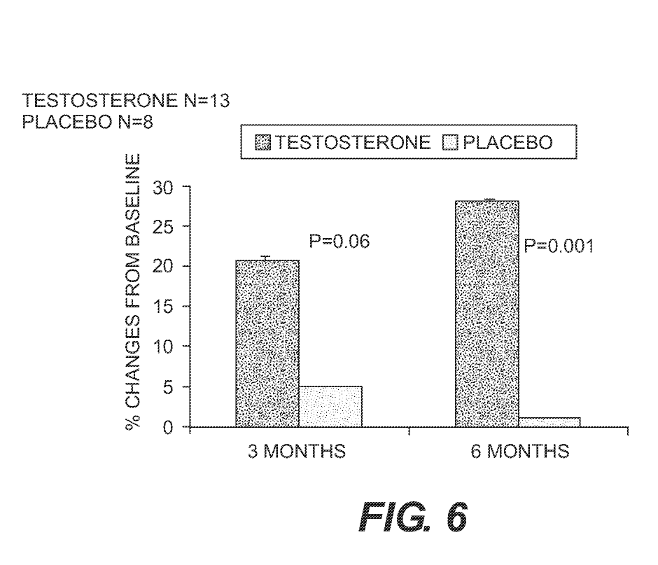

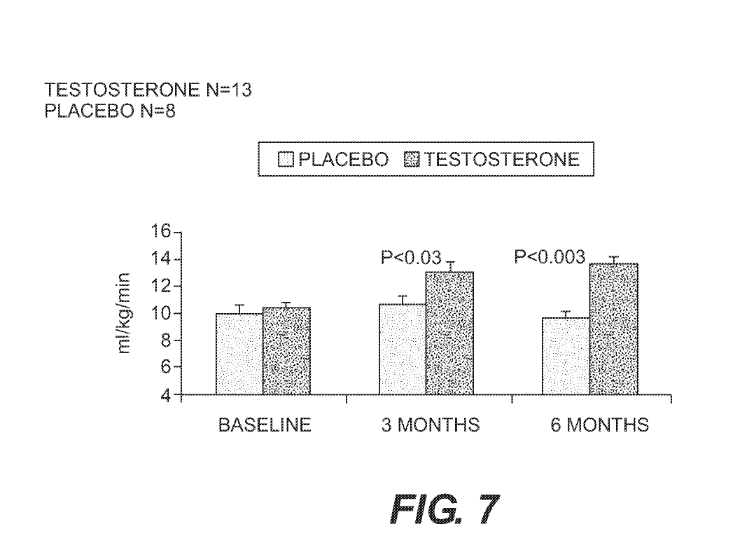

The results show that patients who received testosterone experienced a statistically significant increase in their MVO2, with a p-value of <0.003 for the change within the testosterone group compared to baseline at six months (FIG. 5), and p-values of <0.03 and <0.003 for the changes shown by the testosterone group as compared to the changes in the placebo group at three and six months, respectively (FIG. 7). The results further demonstrate an upwards trend to a statistically significant increase in percent change in MVO2, with a change from baseline of over 25% at 6 months (p-value of 0.001) for patients who received testosterone, as compared to those who received placebo (FIG. 6).

### Example 2

### Muscle Strength

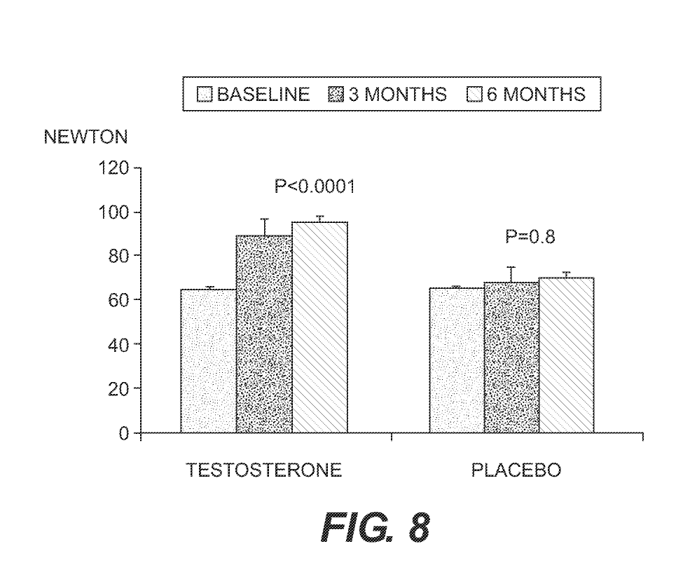

Isometric strength was the highest force developed by patients in three, 5-second maximal voluntary contractions (“MVC”) separated by 1 minute of rest. The knee angle was fixed at 80° (full extension considered 0°). Results are reported at Tables 9-10 and FIGS. 8-10.

TABLE 9

| | Testosterone | Placebo |
|---|---|---|
| baseline | 64.85 N | 65.41 N |
| 3 months | 89.4 N | 67.9 N |
| 6 months | 95.7 N | 69.9 N |

TABLE 10

| | 3 months | 6 months |
|---|---|---|
| Testosterone | 37.8% change | 47.6% change |
| Placebo | 3.87% change | 6.8% change |

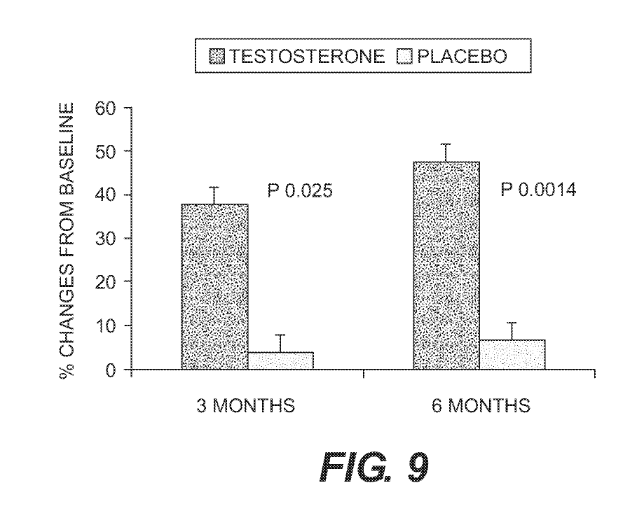

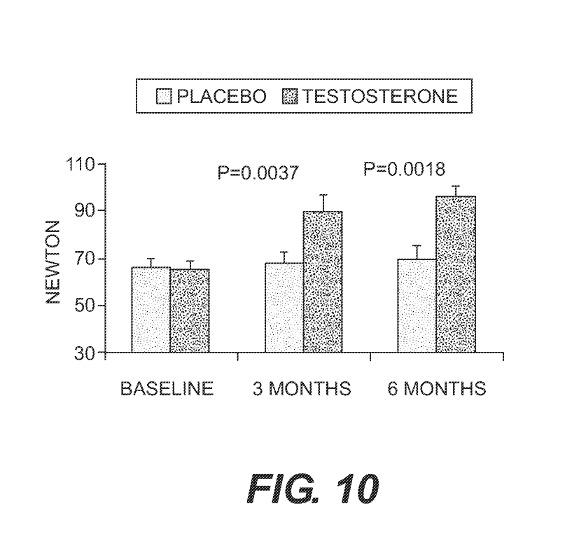

The results demonstrate that patients who received testosterone achieved a statistically significant increase in MVC, with a p-value of <0.0001 at six months of treatment as compared to baseline within the testosterone group (FIG. 8), and p-values of 0.0037 and 0.0018 for the changes shown by the testosterone group as compared to the changes in the placebo group at three and six months, respectively (FIG. 10). The results further demonstrate a statistically significant increase in percent change in MVC from baseline at three and six months, with p-values of 0.025 and 0.0014, respectively (FIG. 9), as compared to placebo.

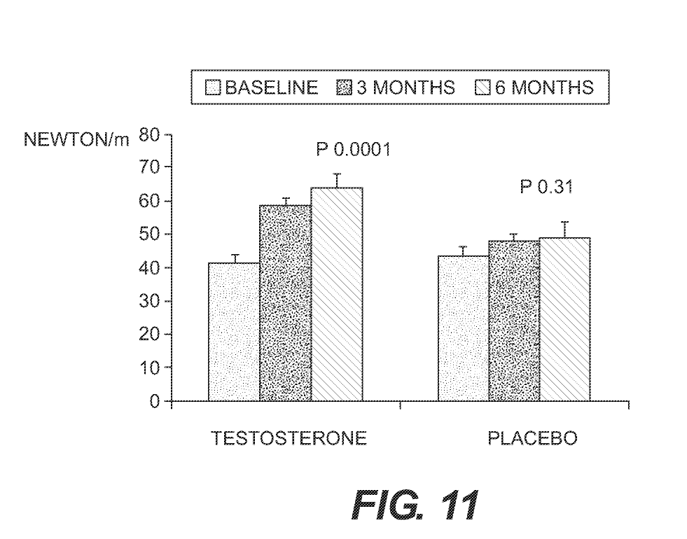

Isokinetic strength was assessed by evaluating the highest peak torque achieved in a five maximal repetition test of concentric knee extension/flexion performed at 90 degree/sec. The range of motion was 50°. Results are reported at Tables 11-12 and FIGS. 11-13.

TABLE 11

| | Testosterone | Placebo |
|---|---|---|
| baseline | 41.35 N/m | 43.66 N/m |
| 3 months | 58.85 N/m | 48.1 N/m |
| 6 months | 63.82 N/m | 49.1 N/m |

TABLE 12

| | Testosterone | Placebo |
|---|---|---|
| 3 Months | 42.32% change | 10.1% change |
| 6 Months | 54.34% change | 12.4% change |

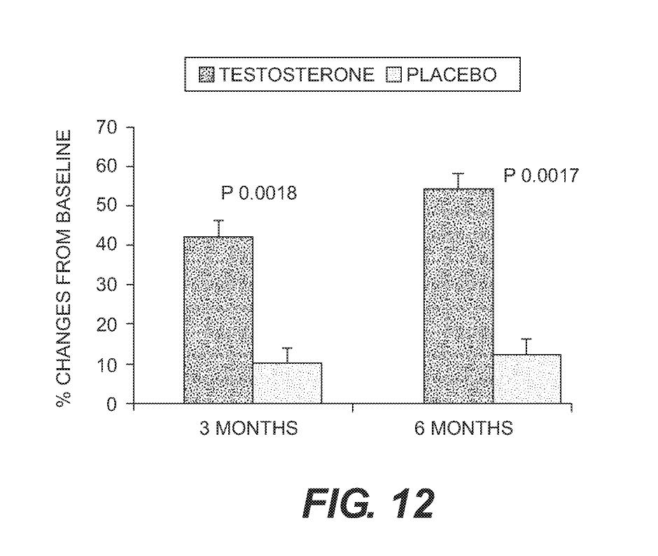

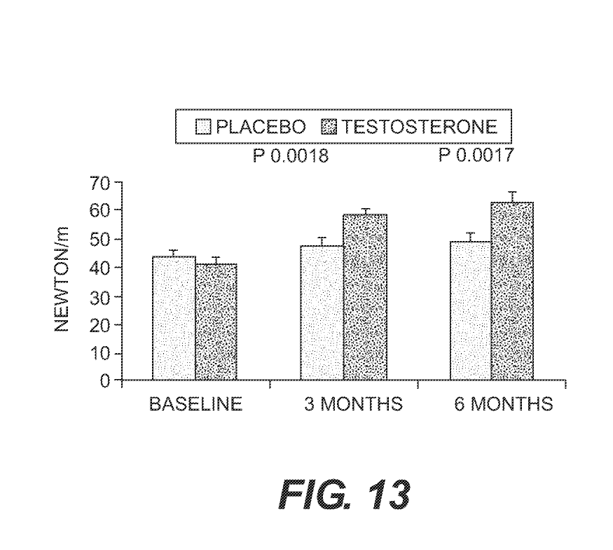

The results show a statistically significant increase in peak torque achieved by patients who were treated with testosterone as compared to baseline, with a p-value of 0.0001 at six months (FIG. 11), and p-values of 0.0018 and 0.0017 for the changes shown by the testosterone group as compared to the changes in the placebo group (FIG. 13). A statistically significant increase in percent changes from baseline occurred at three and six months in patients who were treated with testosterone as compared to placebo, with p-values of 0.0018 and 0.0017, respectively (FIG. 12).

### Example 3

### NYHA Classification Improvement

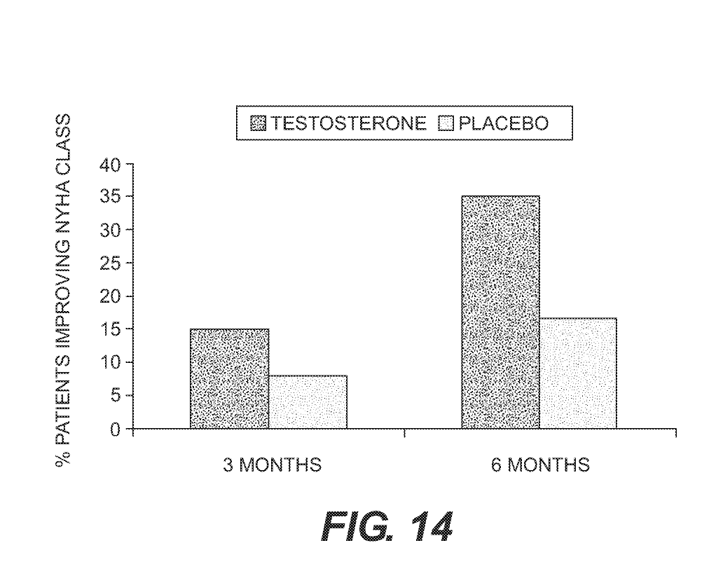

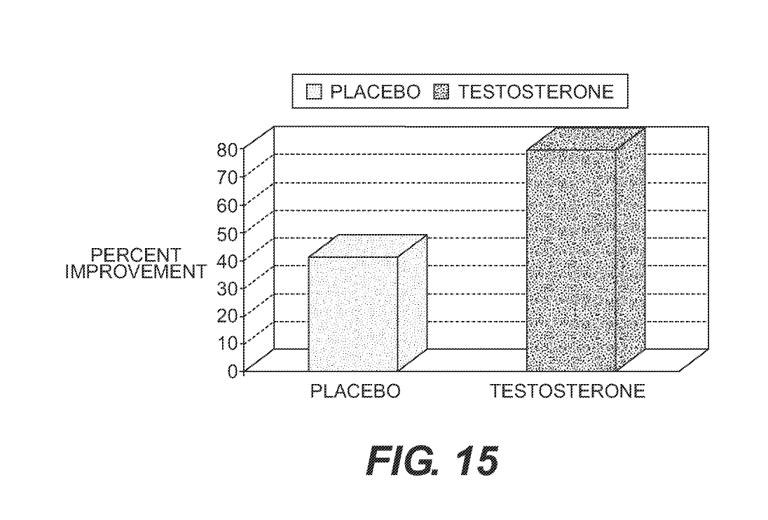

NYHA Classification was determined for each individual at three months and at six months of treatment. The results show that 35% of patients treated with testosterone could be classified in NYHA Class II as compared to baseline, at six months of receiving treatment (Table 13; FIG. 14). This represents a marked improvement in the overall heart failure condition in individuals treated with testosterone. FIG. 15 demonstrates the symptomatic and functional capacity of patients at six months of treatment with testosterone, by showing improvement of NYHA Class and/or improvement of >15% 6MWT, as compared to placebo (Table 14).

TABLE 13
Percent of patients who shifted to NYHA Class II.

| | 3 months | 6 months |
|---|---|---|
| Testosterone | 15% patients | 35% patients |
| Placebo | 8% patients | 16.6% patients |

TABLE 14
Percent of patients with improvement of NYHA
Class and/or improvement of >15% 6 MWT.

| | Placebo | Testosterone |
|---|---|---|
| % Response | 41% | 80% |

TABLE 15
Percent change in NYHA Class of patients
during 6 months of treatment.

| | Placebo | Testosterone |
|---|---|---|
| NYHA − 1 | 20% | 38.8% |
| NYHA 0 | 60% | 50% |
| NYHA + 1 | 20% | 11% |

### Example 4

### Insulin Resistance

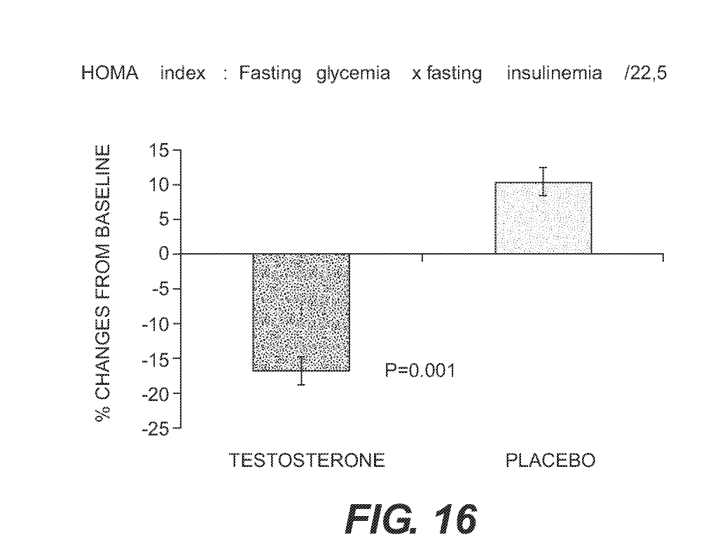

Measurement of insulin resistance was determined for each individual in the testosterone and placebo groups at baseline and then at six months. The measurement was based on fasting plasma glucose and fasting insulin calculated according to the homeostasis model assessment of insulin resistance (“HOMA index”). The results demonstrate a decrease of 16.8% in the amount of insulin resistance within patients who received testosterone for six months, while those who received placebo increased 105% compared to respective baseline values (Table 16; FIG. 16). This demonstrates a beneficial improvement in the metabolism of glucose within patients who received testosterone.

TABLE 16
Percent change from baseline in HOMA index at end of 6 months.

| | Testosterone | Placebo |
|---|---|---|
| % change | −16.8% change | 10.5% change |

### Example 5

### Endothelial-Dependent Vasodilation

Endothelial function (“EndF”) was assessed in one patient in the testosterone group by measuring the change in blood volume and flow in reaction to a brief period ischemia. The reactive hyperemia is a compensatory increase in blood flow caused by local vasodilation of a tissue that was subjected to a short period of ischemia. This event is mediated by endothelium derived nitric oxide (NO), the main mediator of endothelial modulated vasodilation. EndF was evaluated by plethysmographic technique by measuring peripheral arterial tone (Endo-path Itamar Medical, Israel). A blood pressure cuff was placed around the non-dominant arm (study arm) while the other arm served as a control (control arm). The system used a finger probe to assess digital volume changes accompanying vasodilation and increase in blood flow. After a 10-minute stabilization period, the blood pressure cuff was inflated to 50 mmHg above the individual's systolic blood pressure to stop blood flow for 5 minutes. Then the cuff was deflated quickly, allowing reperfusion, while the signal recording continued for 10 minutes. This is a non-invasive technique to assess the vasodilatory capacity (blood flow reserve), which is affected in patients with heart failure.

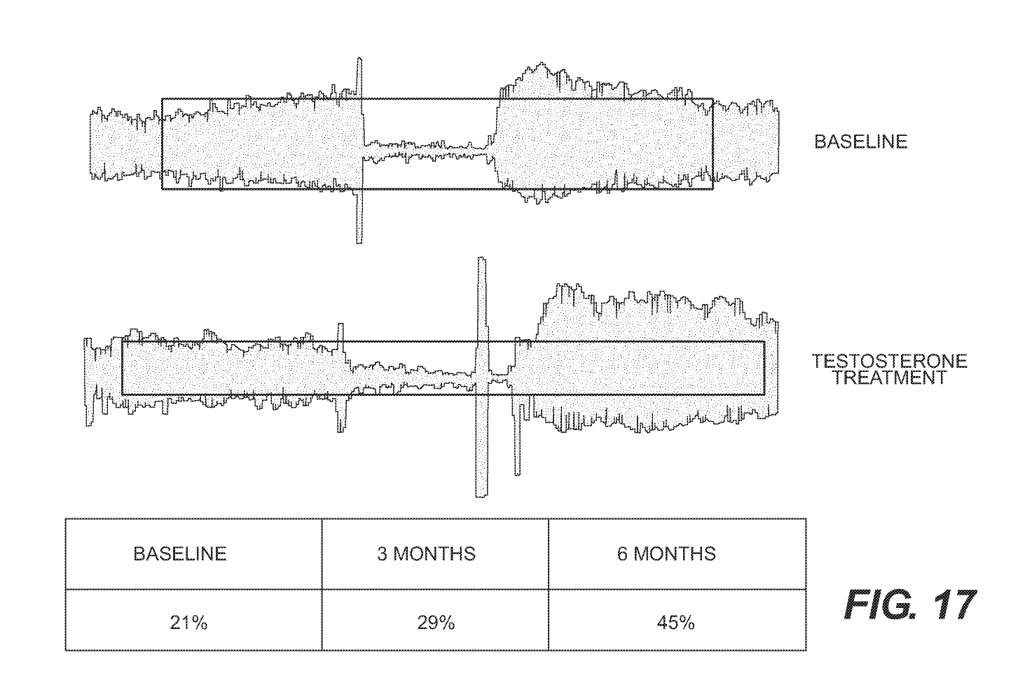

The results show that the patient who received testosterone had a higher index of vasodilation in response to ischemia (from 21% at baseline to 45% after six months) after treatment with testosterone (FIG. 17). This suggests that testosterone can improve blood flow reserve.

### Example 6

### High-Density Lipoprotein

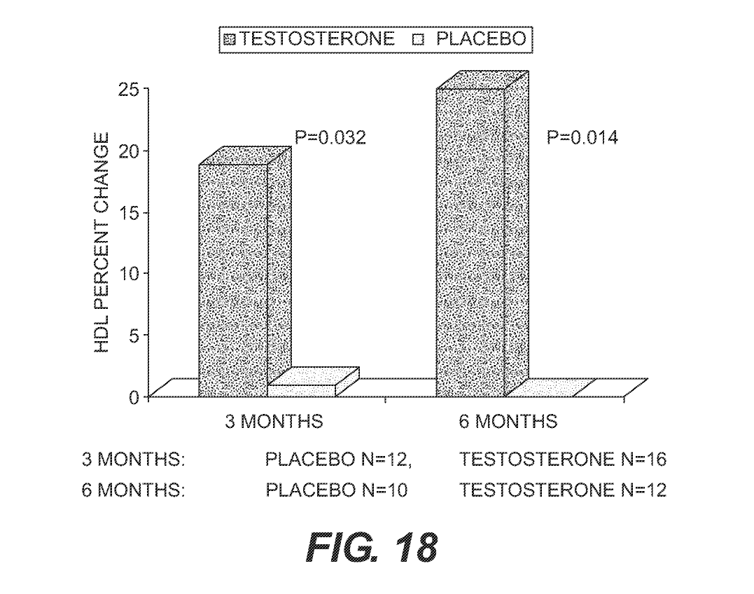

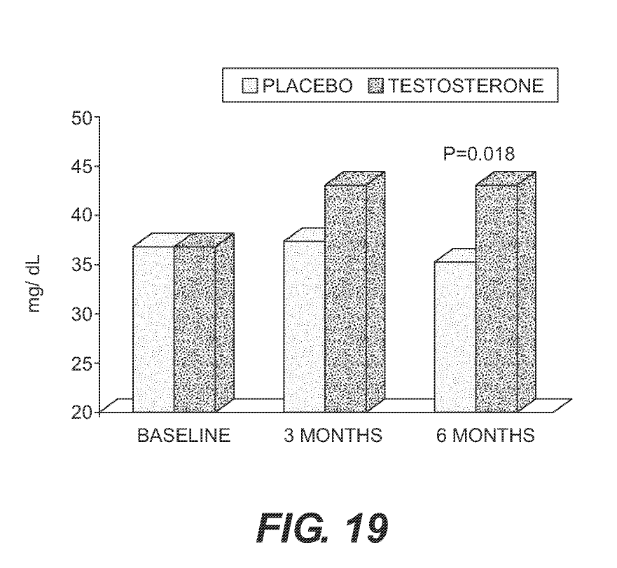

High-density lipoprotein (“HDL”) was measured. The results show a statistically significant increase in HDL percent change, as compared to change in the placebo group, at three and six months by patients who were treated with testosterone, with p-values of 0.032 and 0.014, respectively (FIG. 18; Table 17). A statistically significant increase in the level of HDL was seen in patients at six months as compared to placebo, with a p-value of 0.018 (FIG. 19; Table 18).

TABLE 17
HDL Percent Changes.

| | 3 Months | 6 Months |
|---|---|---|
| Testosterone | 19% | 25% |
| Placebo | 1% | 0% |

TABLE 18
Effect on HDL Levels.

| | Placebo | Testosterone |
|---|---|---|
| Baseline | 36.9 mg/dL | 36.9 mg/dL |
| 3 Months | 37.33 mg/dL | 43.24 mg/dL |
| 6 Months | 35.3 mg/dL | 43.15 mg/dL |

During the clinical study, two patients dropped out of the testosterone group, and one patient dropped out of the placebo group. Of the two patients who dropped out of the testosterone group, one patient referred generalized prurigo (without lesions on the skin) during treatment and discontinued treatment after 3 weeks. The other patient discontinued treatment, without referring any side effect, and based on a suggestion by the patient's doctor of a potential health problem with a patch. The patient who dropped out of the placebo group was a no-show at the 6-week meeting and was reported to have stopped the treatment. Of the testosterone group, six patients referred mild skin irritation at the patch site and continued therapy. Of the placebo group, two patients referred mild skin irritation at the patch site and continued therapy. Two patients in the testosterone group had one episode of worsening of heart failure during the follow-up, which required hospital admission. Both patients, 78 and 82 years old, respectively, had a previous history of very frequent hospital admissions for heart failure before enrolling in the study. These two patients continued the testosterone treatment. Two patients in the placebo group had worsening heart failure that required hospital admission (one patient had one episode of worsening heart failure; the other patient had two episodes of worsening heart failure). These two patients continued with the patch.

While this invention has been particularly shown and described with references to embodiments thereof, it will be understood by those skilled in the art that various changes in form and detail may be made therein without departing from the scope of the invention encompassed by the claims. Such various changes that will be understood by those skilled in the art as covered within the scope of the invention include, in particular, methods directed to administering androgens other than testosterone and administering SARMs.

What is claimed as new and desired to be protected by Letters Patent of the United States is:

1. A method for treating heart failure or for slowing or ceasing the progression of heart failure in a human female, which comprises administering to said female having heart failure a pharmaceutically effective amount of an androgen.

2. A method for treating heart failure or for slowing or ceasing the progression of heart failure in a human female, which comprises administering to said female having heart failure a pharmaceutically effective amount of a selective androgen receptor modulator.

3. The method of claim 1, wherein said androgen is testosterone, dihydrotestosterone (DHT), androstenedione, or methyl testosterone.

4. The method of claim 3, wherein said androgen is testosterone.

5. The method of claim 4, wherein said testosterone is administered in an amount that provides a testosterone serum level concentration of from approximately 2 ng/dL to approximately 2000 ng/dL.

6. The method of claim 5, wherein said testosterone is administered in an amount that provides a testosterone serum level concentration of from approximately 10 ng/dL to approximately 500 ng/dL.

7. The method of claim 6, wherein said testosterone is administered in an amount that provides a testosterone serum level concentration of from approximately 15 ng/dL to approximately 200 ng/dL.

8. The method of claim 5, where said testosterone is administered using a transdermal patch comprising a polymer matrix and said testosterone.

9. The method of claim 8, wherein said patch provides an amount of testosterone of approximately 300 mcg/day.

10. The method of claim 8, wherein said patch comprises approximately 8.4 mg of testosterone.

11. The method of claim 1, wherein said heart failure is associated with any of ACA/AHA Stages B, C or D.

12. A method for improving coronary artery disease, hypertension, dilated cardiomyopathy, valvular heart disease, endothelial function or a disorder of the left ventricle, pericardium, myocardium, endocardium or great vessels in a human female, which comprises administering to said female a pharmaceutically effective amount of an androgen, wherein said human female has heart failure.

13. The method of claim 12, wherein said method is for improving coronary artery disease, hypertension or dilated cardiomyopathy.

14. A method for improving insulin resistance or diabetes in a human female, which comprises administering to said female a pharmaceutically effective amount of an androgen and/or a selective androgen receptor modulator, wherein said human female has heart failure.

15. A method for improving exercise tolerance or NYHA heart failure classification in a human female, which comprises administering to said female a therapeutically effective amount of an androgen and/or a selective androgen receptor modulator, wherein said human female has heart failure.

16. The method of claim 15, wherein said androgen is testosterone.

17. A method for treating heart failure or for slowing or ceasing the progression of heart failure in a human female having one or more structural heart changes, which comprises administering to said female having said one or more structural heart changes a pharmaceutically effective amount of an androgen.

18. The method of claim 17, wherein said androgen is testosterone.

19. A method for treating heart failure or for slowing or ceasing the progression of heart failure in a human female having one or more structural heart changes, which comprises administering to said female having said one or more structural heart changes a pharmaceutically effective amount of testosterone, wherein said testosterone is administered in an amount that provides a testosterone serum level concentration of from approximately 80 ng/dL to approximately 100 ng/dL.

20. A method for treating heart failure or for slowing or ceasing the progression of heart failure in a human female, which comprises administering to said female having heart failure a pharmaceutically effective amount of an androgen and a selective androgen receptor modulator.

21. The method of claim 20, wherein said androgen and selective androgen receptor modulator are administered in an amount that provides a therapeutic equivalence to a testosterone serum level concentration of from approximately 2 ng/dL to approximately 2000 ng/dL.
